(19) United States
(12) Patent Application Publication
(10) Pub. No.: US 2010/0120013 A1
(43) Pub. Date: May 13, 2010

(54) PROCEDURE FOR LONG TERM CORNEAL CULTURE

(75) Inventors: Daniel R. Cerven, Quakertown, PA (US); Michelle A. Piehl, Emmaus, PA (US); Albert C. Gilotti, Bethlehem, PA (US); Alison L. Donovan, Quakertown, PA (US); George L. DeGeorge, Allentown, PA (US); Micheal R. Carathers, Allentown, PA (US)

Correspondence Address: PAUL AND PAUL, 2000 MARKET STREET, SUITE 2900, PHILADELPHIA, PA, 19103, US

(73) Assignee: MB RESEARCH LABORATORIES, INC., Spinnerstown, PA (US)

(21) Appl. No.: 12/266,906

(22) Filed: Nov. 7, 2008

(51) Int. Cl. A01N 1/02; C12Q 1/02

(52) U.S. Cl. 435/1.1; 435/29

(57) ABSTRACT

A method for long-term multi-species cornea culture and preservation is provided. Excised mammalian corneas are preserved with structural integrity by filling the endothelial cavity with a plug material. Plugged corneas are sterile-cultured for more than 21 days via an air-interface culture where nutrients from a culture media are supplied to corneal tissue primarily through the plug. Plugged corneas are incubated in culture dishes with a customized media continuously in contact with the ocular sclera and corneal plug, and periodically bathing the corneal surface epithelia. An animal-alternative toxicology assay is also provided using excised porcine corneas capable of assessing ocular injury reversibility within 21 days. Further provided is a method of mammalian corneal preservation for excised human corneas extending their storage life to three to four weeks.

### BACKGROUND OF THE INVENTION

This invention relates to the maintenance of excised mammalian corneas in a living state for an extended period of time in order to serve as a model system for experimental testing.

The invention also relates to the use of a culture system in conducting a specific porcine corneal damage and reversibility toxicology test that spans extended periods of time.

This invention further relates to using a culture system to prolong the maximum preservation time for human corneas slated for human corneal transplantation surgery.

An expansion of ocular surgical techniques and the need for donor ocular tissues has yielded a demand for increasing the quantity and the quality of preserved ocular tissues, especially corneal tissues. Delays in time, from donor to recipient, caused by transportation and donor health screens need to be compensated for, indicating a need for long-term corneal storage. Due to the nature of the fragile external and internal cellular layers of the cornea, the epithelia and endothelia respectively, careful maintenance of the corneal morphology is imperative.

An excised cornea is most useful for toxicological testing, and in addition for implantation, when the tissue isolated from a donor, whether human or other mammalian, is preserved in such a manner so as to maintain maximum physiological function.

Thus, there is a critical need for a robust method for excised corneal storage and culturing so as to maintain normal in vivo corneal performance until a recipient transplantation can occur.

Regulatory agencies require manufacturers of products to characterize product eye irritation/damage risk, and mandate the use of animals. The Draize test for acute toxicity was devised by the Federal Food and Drug Administration, in 1944, for conducting such tests for up to 14 days using live rabbits. Eye irritation is still characterized using a live rabbit test.

The Draize rabbit eye test, as currently used, evaluates the effect of a test substance on eye tissues for a period of three weeks. This extended time period allows the evaluation of reversibility of any damage caused by the introduced toxins. However, due to the ethical issues involved in the testing of animals, there is an effort to modify current practices in toxicology to reduce, refine and replace the number of animals currently involved in product safety testing with other reliable testing methods.

Although there are several non-animal alternative methods to characterize aspects of eye irritation and damage, no established method can currently model recovery after injury as in a Draize test. At the root of this problem has been the lack of longevity in cadaver corneal culture.

Some regulatory classification methods of ocular irritancy require a healing time factor which extends the mandated length of the test to 21 days. The most important parameters in the regulatory regulations regarding this testing are the extent of corneal opacity (injury) and its reversibility. In fact, corneal opacity can be responsible for up to 73% of the total score in calculations of maximum average eye irritation scores.

Corneal opacity may be visualized by fluorescein staining in the Draize eye test. As the opacities clear in vivo, the eyes receive a lower score based on the area of the opacities. A similar phenomenon can be implemented to measure ocular injury and reversibility in excised and cultured mammalian corneas.

For the purposes of toxicological testing and for purposes of corneal preservation for implantation, robust techniques for the preservation of corneal tissue integrity and viability after excision from living organisms are critical and necessary.

Excision of mammalian corneas for several purposes indicates separation of the cornea from attached tissue while retaining a rim of sclera to support the fragile corneal tissue.

There is a need for a method to maintain the physical corneal shape and structure, and also to deliver nutrients to all layers of the cornea including the layers of vital importance, i.e., the surface epithelial and basal endothelial cell layers. There is also a need to address the preservation of the vitality of cornea tissue for various ends, i.e. toxicological testing or implantation.

It is an objective of the present invention to provide mammalian corneal culture preservation procedure, components, and toxicological ocular irritancy test with enhanced results, wherein the cornea are robustly maintained for in excess of 21 days.

### SUMMARY OF THE INVENTION

The present invention provides a method for long-term mammalian corneal storage and preservation, i.e., for a period comfortably in excess of 21 days.

Also provided is a specific preservation agar/gelatin mixture, a customized culture media, and a special mechanical “blinking” simulation using a precisely timed rocker platform.

Further provided is a method for assessment of ocular toxicity and recovery in excised cultured porcine corneas.

Moreover, the present invention applies the corneal culture method to human corneas excised from corneal donors in order to enhance the preservation of donor corneas until recipient transplant can occur.

Excised mammalian corneas are preserved with structural integrity by filling the endothelial cavity with an agar/gelatin plug. Plugged corneas are sterile-cultured for more than 21 days, and up to four weeks, via an air-interface culture technique where nutrients from culture media are supplied to corneal tissue primarily through the agar plug.

The cultured corneas are incubated in tissue culture dishes with customized media that remains in contact with the ocular sclera and corneal plug continuously and bathes the corneal surface epithelia periodically.

An animal-alternative toxicology assay is employed using excised porcine corneas, which is capable of assessing ocular injury reversibility within 21 days.

Further included is a method of mammalian corneal preservation to be applied to human corneas to extend their storage life up to three to four weeks, allowing greater potential for successful use in transplantation.

The invention includes a method for mammalian corneal excision from whole globe eyes, FIG. 5, which comprises:

procuring enucleated whole globe mammalian eyes (11) from an abattoir and transporting said eyes on ice in an isotonic buffered saline solution such as Hank's Balanced Salt Solution (HBSS) supplemented with an anti-fungal drug such as approximately 5 μg/ml Amphotericin B;

incubating whole globe eyes in a broad spectrum antiseptic such as 1% Povidone-iodine for approximately 2 minutes in a sterile field (13);

briefly rinsing said eyes with an isotonic buffered saline solution (15) such as dPBS (Dulbecco's phosphate buffered saline solution), and then incubating said eyes with an amino glycoside antibiotic such as 1 mg/ml gentamicin in dPBS for 15 minutes (17);

excising the cornea from the eye with a scalpel using sterile technique, by making an incision 2-3 mm from the cornea into the sclera and cutting at this same distance all around the cornea until the cornea is free from the eye and removing the iris from the cornea with a pair of forceps and discarding said iris (19);

rinsing the cornea in a series of 12 sterile HBSS baths (21) and storing said corneas in HBSS at room temperature until mounted/needed (23); and

discarding any unacceptable corneas.

### DESCRIPTION OF THE DRAWINGS

The features, advantages and operation of the present invention will become readily apparent and further understood from a reading of the following detailed description with the accompanying drawings, in which like numerals refer to like elements, and in which:

FIG. 1 is a histological cross-section of a hematoxylin and eosin stained porcine cornea that demonstrates the viability of the corneal tissue after 21 days in culture according to the invention;

FIG. 2 is an image demonstrating several inverted corneal endothelial cavities in the process of being filled with the agar/gelatin mixture to preserve the structural integrity of the corneal morphology according to the invention;

FIG. 3 shows four agar/gelatin filled corneas in a culture dish with media according to the invention;

FIG. 4 shows sodium fluorescein (NaFL) staining of a test material damaged cultured cornea as per conduction of the toxicological ex vivo ocular irritancy and reversibility test method according to the invention;

FIG. 5 shows the basic process steps for preparing cornea for use in toxicological testing;

FIG. 6 shows the basic process steps for preparing and incubating plugged cornea for an extended period of time;

FIG. 7 shows the basic process steps for performing toxicological testing; and

FIG. 8 shows the basic process steps for preparing, storing and maintaining corneas for transplant.

### DETAILED DESCRIPTION OF THE INVENTION

The invention provides methods and compositions for sterile excision of mammalian corneas from whole globe eyes, long term corneal culture of said excised corneas, a toxicological ex vivo ocular irritancy and reversibility test that can be performed subsequently on said cultured corneas, and an application for using the corneal culture system to preserve human corneas for transplantation.

Substances used in the invention include:

a) A buffered saline solution, such as Hanks' Balanced Salt Solution (HBSS);
b) A broad spectrum antibiotic, such as penicillin-streptomycin;
c) An anti-fungal drug, such as Amphotericin B (Amp B);
d) An amino glycoside antibiotic, such as Gentamicin;
e) A broad spectrum antiseptic, such as Povidone-iodine (PVPI);
f) An isotonic wash buffered saline solution, such as dPBS (Dulbecco's phosphate buffered saline solution);
g) Ultra-pure sterile water, such as tissue culture water;
h) A saline-based isotonic cell culture media, such as M199 culture media;
i) A globular protein serum solution, such as fetal bovine serum (FBS);
j) A salt, such as sodium bicarbonate (NaHCO₃);
k) An amino acid, such as L-glutamine;
l) A fluorescent tissue stain, such as sodium fluorescein (NaFL);
m) A gelatinous substance, such as Agar; and
n) An additional gelatinous substance, such as porcine skin gelatin.

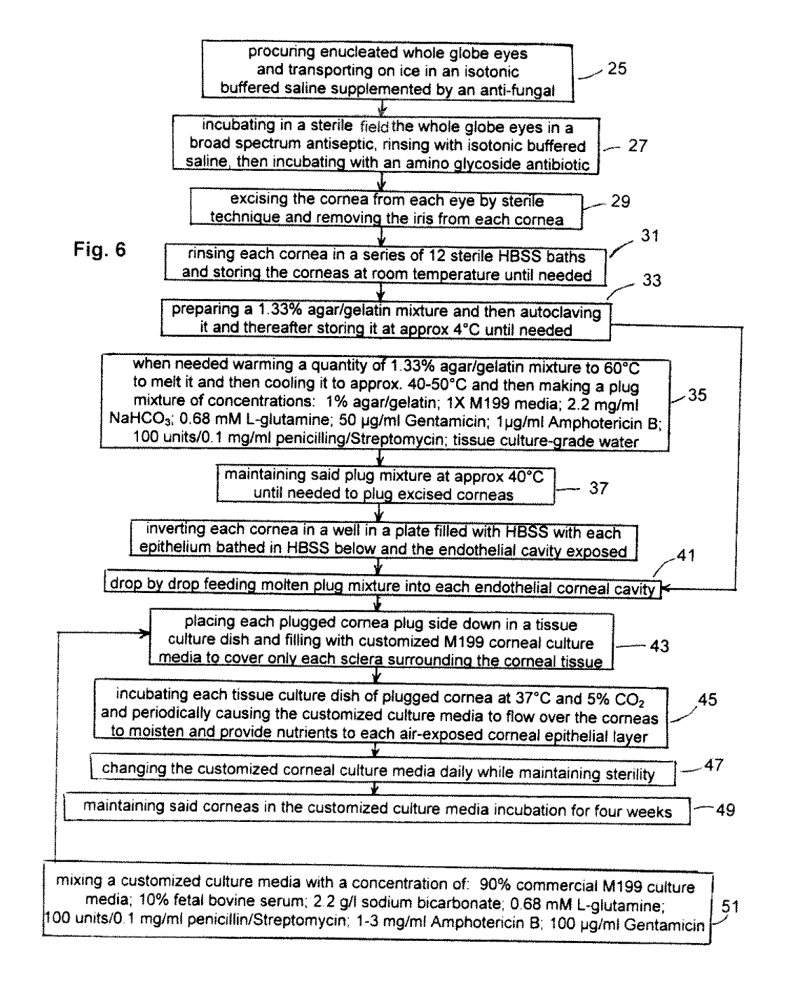

A first embodiment of the present invention is directed to a method for mammalian corneal excision from whole globe eyes, and long-term mammalian corneal storage and preservation for greater than 21 days, to include a specific preservation culture media. At 21 days the method yields a histologically normal cross section of a cornea, FIG. 1. The corneal culture process of the invention, FIG. 6, includes the following steps which are all conducted using sterile techniques in order to maintain sterility of the corneas in culture.
a) Enucleated mammalian whole globe eyes are procured from an abattoir and transported on ice in an isotonic buffered saline solution such as Hank's Balanced Salt Solution (HBSS) supplemented with an anti-fungal drug (25) such as Amphotericin B (approximately 5-μg/ml final concentration).
b) In a sterile field, whole globe eyes are incubated in approximately 1% Povidone-iodine for approximately 2 minutes, briefly rinsed with Dulbecco's Phosphate Buffered Saline (dPBS), and then incubated with approximately 1 mg/ml Gentamicin in dPBS for 15 minutes (27).
c) Using sterile technique, the cornea is excised from the eye with a scalpel by making an incision 2-3 mm from the cornea into the sclera and cutting at this same distance all around the cornea until the cornea is free from the eye. The iris is removed from the cornea with a pair of forceps and discarded (29).
d) The cornea is then rinsed in a series of 12 sterile HBSS baths and stored in HBSS at room temperature until mounted (31). Corneas that are determined to be unacceptable are discarded.
e) A 1.33% agar/gelatin mixture is prepared in ultra pure sterile water, sterilized by autoclaving and stored at approximately 4° C. (33). Before beginning corneal excision, the 1.33% agar/gelatin mixture is warmed to approximately 60° C. to melt it, and then cooled to approximately 40-50° C. Thereafter, the following components are added to the agar/gelatin: 10× M199 corneal culture media, 75 mg/ml NaHCO3, 200 mM L-glutamine, 50 mg/ml Gentamicin, 250 μg/ml Amphotericin B; and 100× penicillin/Streptomycin (35) to arrive at a corneal plug mixture with the following final concentrations.

| | Final |
|---|---|
| Component | Concentration |
| 1.33% agar/gelatin | 1% |
| 10X M199 (commercial grade) | 1X |
| 75 mg/ml NaHCO₃ | 2.2 mg/ml |
| 200 mM L-glutamine | 0.68 mM |
| 50 mg/ml Gentamicin | 50 ug/ml |
| 250 ug/ml Amphotericin B | 1 μg/ml |
| 100X Penicillin/Streptomycin | (100 units/0.1 mg)/ml |

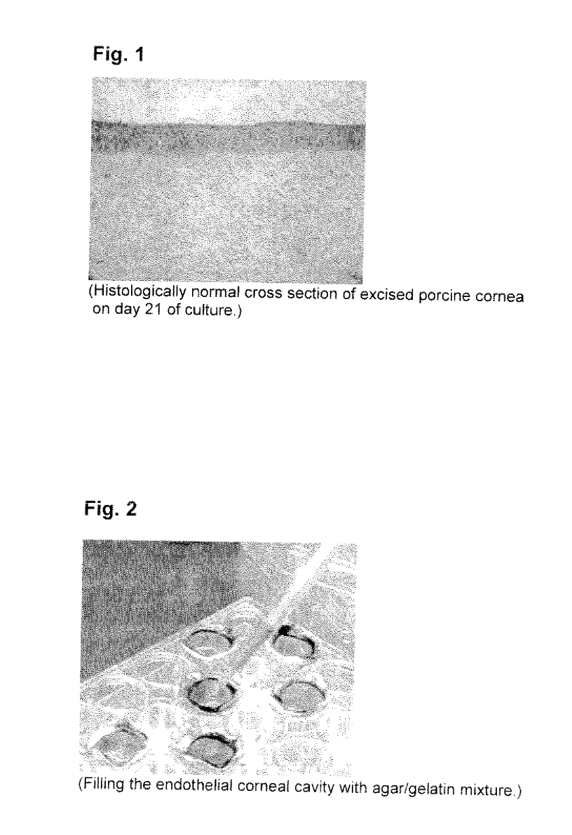

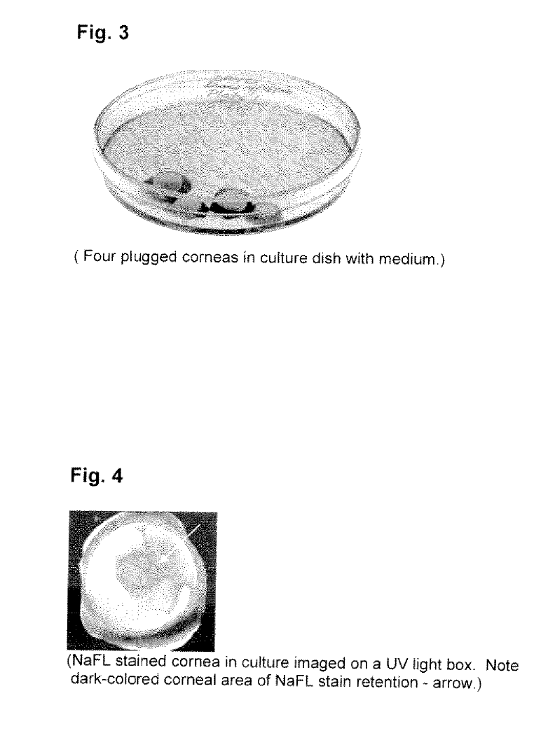

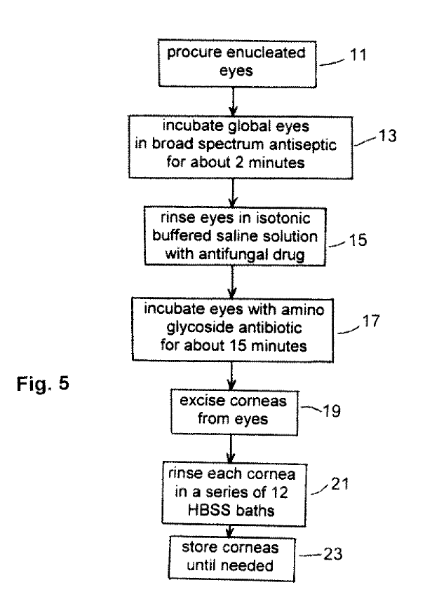

The final concentration of the agar/gelatin in the plug mixture is 1% (a dilution from 1.33% to 1%).
f) Plug mixture is maintained at approximately 40° C. until all corneas are excised and ready to be plugged with plug mixture (37).
g) Corneas are inverted atop one well from a 24-well plate filled with HBSS with one cornea per well, so that the epithelium is bathed in HBSS below, and the endothelial cavity is exposed and able to be filled with the warmed molten agar/gelatin mixture at approximately 40° C. (39).
h) The warmed molten plug mixture is added drop by drop into the exposed endothelial cornea cavity, and then allowed to coot and congeal (41). This process is continued until the cavity is completely filled with congealed plug mixture and supports the natural curvature of the cornea in culture, FIG. 2.
i) The plugged corneas are then placed agar/gelatin plug side down in a 150×25 mm tissue culture dish and the dish is filled with a customized M199 corneal culture media to cover the sclera tissue surrounding the corneal tissue (43), but not the corneal tissue itself, FIG. 3. The exposed corneal epithelial layer of the cornea must remain able to interface with the air in culture within the sterile culture dish.
j) Dishes of corneas are placed into incubator set to 37° C. and 5% CO₂. Dishes are placed on a special rocker that periodically tilts to 45° causing the culture media to flow over corneas in culture to moisten and provide nutrients to the air-exposed corneal epithelial layer of cells (45).
k) For the duration of the corneal culture period, the corneal culture media is changed daily using a sterile technique (47) by removing used, old corneal culture media from each culture dish by sterile aspiration and by replacing it with pre-warmed (37° C.) fresh corneal culture media. This permits the corneas to be maintained for up to four weeks (49).

Also provided is a formulation for a custom preservation corneal culture media (51). This customized corneal culture media departs from commercially available M199 culture media which is formulated with either Earle's salts or Hank's salts.

The invention uses a customized M199 corneal culture media, where in the preferred formulation the components are:

1. Tissue culture water (SIGMA W3500)

2. 10× M199 culture media (INVITROGEN 11825)

3. Sodium bicarbonate (NaHCO₃; SIGMA S5761)

4. L-glutamine (INVITROGEN 25030)

5. Fetal bovine serum (FBS)

6. Penicillin/Streptomycin (INVITROGEN 15140)

7. Amphotericin B (Amp B; SIGMA A2942)

8. Gentamicin (BIOWHITAKER BW17-518L)

The concentration of components in the customized M119 corneal culture media is as follows.

| Component | Volume | |
|---|---|---|
| tissue culture water | 779.4 | ml |
| 10X M199 (commercial) | 100 | ml |
| NaHCO₃ | 2.2 | g |
| 200 mM L-glutamine | 3.4 | ml |
| fetal bovine serum | 100 | ml |
| 50 mg/ml Gentamicin | 1-3 | ml |
| 100X Penicillin/Streptomycin | 10-30 | ml |
| 250 μg/ml Amphotericin B | 4-12 | ml |
| Total Volume | 1000 | ml |

To prepare the customized M199 corneal culture media all components are combined, the pH is determined and adjusted with 1 N HCl or 1 N NaOH if necessary to an acceptable range of 7.0-7.4; and the solution is sterilized by passing it through a 0.20 μm filter flask. The resultant prepared media is sterile and is maintained according to sterile techniques, and is capable of being stored at approximately 4° C. for up to two weeks.

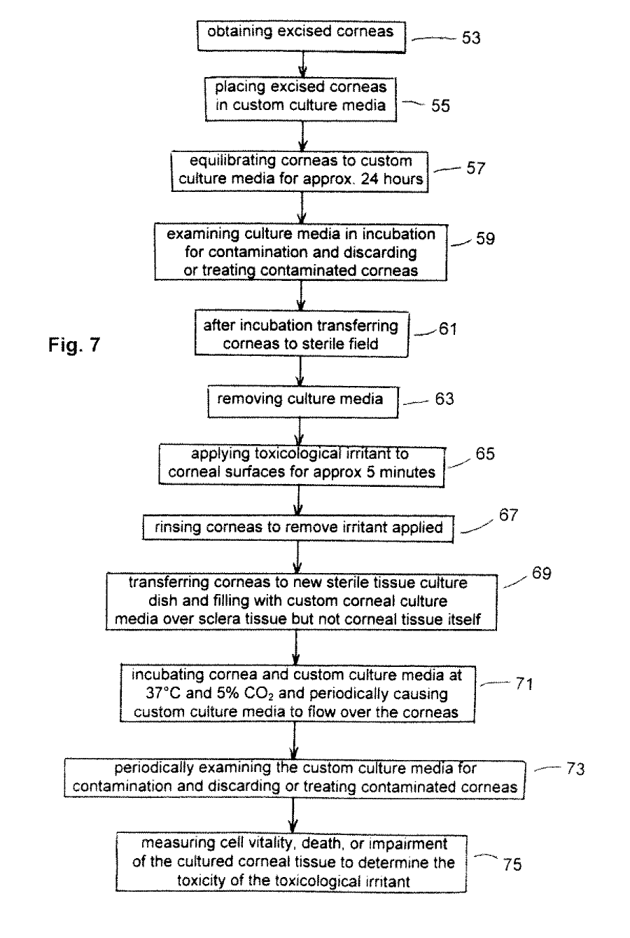

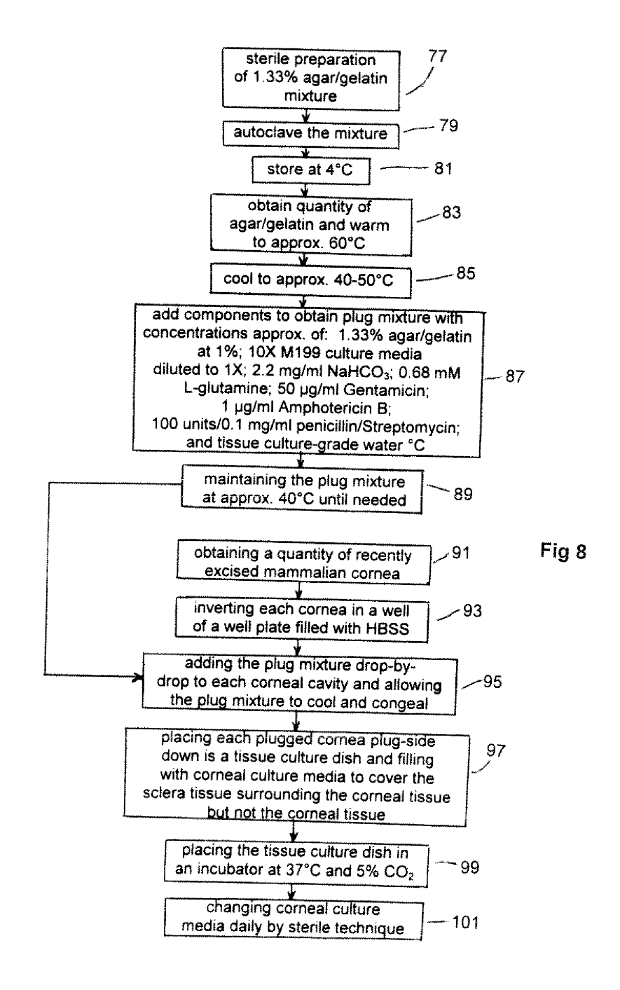

Further provided is a method for toxicological ocular irritation and recovery testing, in excised cultured mammalian corneas. The toxicological ocular irritation and recovery testing procedure is conducted on excised corneas (53), FIG. 7, according to the following steps.
a) The toxicological ocular irritation and recovery testing procedure begins approximately 24 hours after the corneas are excised. This allows the corneas to be placed in a custom culture media (55) to equilibrate to the culture conditions for 24 hours (57). The day of excision is considered day “1”, and the day of dosing is considered day “0” for the purposes of the timing for the toxicological ocular irritation and recovery testing procedure.
b) The customized culture media is examined microscopically for the presence of contamination throughout an incubation process in which the toxicological ocular irritation and recovery testing is conducted. If contamination is evident, the culture dish and corneas are discarded, or treated with additional antibiotics/antifungal drugs in an effort to eliminate the source of contamination. If this proves unsuccessful, the dish and cornea are discarded (59).
c) Cultured corneas are removed from the incubator and transferred to a sterile field (61). For the purpose of the toxicological ocular irritation and recovery testing procedure, the preferred method is four corneas per culture dish and one experimental condition (positive control or negative control) per culture dish.
d) Toxicological dosing of the corneas in the culture dish is performed by removing culture media from the dish (63), applying 10 μl or 20 mg of the testing material directly to the corneal surface; and allowing the corneal tissue to be dosed with the testing material for five minutes (65). Dosing times can be staggered so that each cornea per dish is dosed at time intervals (i.e. 1-minute).
e) After the 5-minute dosing period, individual corneas are gently rinsed with approximately 2 ml of dPBS to remove the testing material (65).
f) Dosing is repeated with additional testing materials, one condition per dish, including two additional control conditions, i.e., 100% concentration ethanol (positive control) and dPBS (negative control).
g) After dosing, each set of corneas is carefully transferred to a new, sterile 150×25 mm tissue culture dish using a disposable cell lifter. The new culture dish containing the dosed corneas is filled with the customized M199 corneal culture media to cover the sclera tissue surrounding the corneal tissue, but not the corneal tissue itself (69). The exposed corneal epithelial layer of the cornea must remain able to interface with the air in culture, within the sterile culture dish.
h) Then the new dishes of dosed corneas are returned to an incubator set to 37° C. and 5%, CO₂. Dishes are placed on a rocker that periodically tilts to 45° causing the culture media to flow over the corneas to moisten and provide nutrients to the air-exposed corneal epithelial layer of cells (71).
i) The culture media is examined microscopically throughout the toxicological ocular irritation and recovery testing procedure for presence of contamination. If contamination is evident, the culture dish and corneas are discarded or treated with additional antibiotics/antifungal drugs. Unsuccessfully treated corneas are discarded (73).
j) The toxicity of the test material is determined by measuring cell vitality, death, or impairment in the cultured corneal tissue via confocal microscopy paired with various vital dyes, whether colored or fluorescent. Or by using reflective confocal microscopy, without dyes. Or by using digital imaging techniques including image processing software. Otherwise, visual inspection combined with a vitality or death marker dye such as sodium fluorescein (NaFL) stain is used (75).

Also further included is a method for measuring cell death or impairment with NaFL in the cultured corneal tissue according to the following steps.
i) On days “1”, “2”, “3”, “7”, “10”, “14”, and “21” of the toxicological ocular irritation and recovery testing procedure, the percentage of damaged corneal tissue area is determined by staining with 2% sodium fluorescein (NaFL) stain as in the Draize rabbit eye test.
ii) The culture media is examined microscopically throughout the toxicological ocular irritation and recovery testing procedure for presence of contamination. If contamination is evident, the culture dish and corneas are discarded or treated with additional antibiotics/antifungal drugs. Unsuccessfully treated corneas are discarded.
iii) Cultured corneas are removed from the incubator and transferred to a sterile field. Sterile 2% NaFL is added drop-wise into each cornea until entire cornea is covered. This step is repeated until all corneas in a dish are covered with the 2% NaFL solution. All corneas are subsequently rinsed gently with dPBS until excess NaFL is no longer present. Each cornea is individually transferred with a disposable cell lifter to a new, sterile culture dish.
iv) NaFL stain will be retained in tissue that is damaged. The corneal areas of stain retention can be observed as a brown color when placing the culture dish containing the NaFL stained corneas on a regular white-light box. Alternatively, the corneas can be placed on a UV light box to observe NaFL stain retention, as shown in FIG. 4. Corneal sterility in culture must be maintained throughout this procedure.
v) Corneal damage is scored by assessing the total area of damage, i.e. the NaFL stain retention in damaged tissue, and then assigning a corneal damage score according to the following scale.
   0=no corneal damage
   1=0 to 25% corneal area damaged
   2=25% to 50% corneal area damaged
   3=50% to 75% corneal area damaged
   4=75% to 100% corneal area damaged
Alternatively, corneal damage area can be determined using digital imaging system and image analysis software such as METAMORPH [Nikon].
vi) Positive control corneas dosed with 100% concentration ethanol no longer retain stain at approximately 3 to 10 days in culture. PBS-treated corneas should no longer retain stain by day “3”. These are the acceptance limits for the test.
vii) A mean corneal recovery score for a cornea “batch” is determined by averaging the time in days when the corneas in the batch no longer retain the NaFL stain and are recovered. Additionally, if all corneas no longer retain stain by day “21”, the test material (toxin) is determined to cause reversible ocular damage. If any corneas retain NaFL stain at day “21”, the test material (toxin) is considered to cause irreversible ocular damage.
k) After the corneal damage is scored, each culture dish is filled with a customized M199 corneal culture media to cover the sciera tissue surrounding the corneal tissue, but not the corneal tissue itself. The exposed corneal epithelial layer of the cornea must remain able to interface with the air in culture within the sterile culture dish.
l) Dishes of corneas are returned to an incubator which is set to 37° C. and 5% CO₂. Dishes are placed on a custom rocker that periodically tilts to 45° causing the culture media to flow over the corneas to moisten and provide nutrients to the air-exposed corneal epithelial layer of cells.
m) The toxicity determination of the test material can be repeatedly measured by determining cell vitality or death or impairment in the cultured corneal tissue at any time during the culture period, which culture period is to be greater than 21 days.

Also further provided is an application for using the corneal culture system to preserve donor human corneas for transplantation into recipients, FIG. 8. This application includes the following steps.
a) Enucleated human whole globe eyes are procured from an eye bank and transported on ice in an isotonic buffered saline solution such as Hank's Balanced Salt Solution (HBSS) supplemented with an anti-fungal drug such as Amphotericin B, having approximately 5-μg/ml final concentration.
b) In a sterile field, whole globe eyes are incubated in approximately 1% Povidone-iodine for approximately 2 minutes, briefly rinsed with dPBS, and then incubated with approximately 1 mg/ml Gentamicin in dPBS for 15 minutes.
c) Using a sterile technique, the cornea is excised from the eye with a scalpel by making an incision 2-3 mm from the cornea into the sclera and cutting at this same distance all around the cornea until the cornea is free from the eye. The iris is removed from the cornea with a pair of forceps and discarded.
d) The cornea is then rinsed in a series of 12 sterile HBSS baths and stored in HBSS at room temperature until mounted. Corneas that are determined to be unacceptable are discarded.
e) A 1.33% agar/gelatin mixture is prepared in ultra pure sterile water (77), FIG. 8. It is then sterilized by autoclaving (79) and thereafter stored at approximately 4° C. (81). Before beginning corneal excision, the 1.33% agar/gelatin mixture is warmed to approximately 60° C. to melt it (83), and then cooled to approximately 40-50° C. (85). Then additional components are added which reduces the concentration of agar/gelatin from 1.33% to 1.0%. These components are: 10× M199 corneal culture media, 75 mg/ml NaHCO3, 200 mM L-glutamine, 50 mg/ml Gentamicin, 250 μg/ml Amphotericin B; and 100× penicillin/strptomycinre. The final mixture is used as a corneal plug mixture with the following final concentrations (87).

| | Final |
|---|---|
| Component | Concentration |
| 1.33% Agar/gelatin | 1% |
| 10X M199 (commercial) | 1X |
| 75 mg/ml NaHCO3 | 2.2 mg/ml |
| 200 mM L-Glutamine | 0.68 mM |
| 50 mg/ml Gentamicin | 50 ug/ml |
| 250 ug/ml Amphotericin B | 1 μg/ml |
| 100X penicillin/Streptomycin | (100 units/0.1 mg)/ml |

f) The agar/gelatin mixture is maintained at approximately 40° C. (89) until all corneas are excised (91) and ready to be plugged with agar/gelatin mixture.
g) Corneas are inverted atop one well from a 24-well plate filled with HBSS (one cornea per well) so that the epithelium is bathed in HBSS below, and the endothelial cavity is exposed and able to be filled with the warmed, molten agar/gelatin mixture held at approximately 40° C. (93).
h) The warmed, molten agar/gelatin mixture is added drop by drop into the exposed endothelial corneal cavity, and then allowed to cool and congeal (95). This process is continued until the cavity is completely filled with congealed agar/gelatin mixture and supports the natural curvature of the cornea in culture, FIG. 2.
i) The agar/gelatin plugged corneas are then placed agar/gelatin plug side down in a 150×25 mm tissue culture dish and the dish is filled with a customized M199 corneal culture media to cover the sclera tissue surrounding the corneal tissue (97), FIG. 8, but not the corneal tissue itself. This is shown in FIG. 3. The exposed corneal epithelial layer of the cornea must remain able to interface with the air in culture within the sterile culture dish.
j) Dishes of corneas are placed into an incubator set to 37° C. and 5% CO₂. The dishes are placed on a special rocker that periodically tilts to 45° causing the culture media to flow over the corneas in the culture to moisten and provide nutrients to the air-exposed corneal epithelial layer of cells (99).
k) For the duration of the corneal culture period, the custom corneal culture media is changed daily using a sterile technique by removing used, old corneal culture media from each culture dish, i.e. by sterile aspiration, and by replacing the culture with pre-warmed (37° C.) fresh corneal culture media (101)
l) When needed for transplant, corneas are shipped on ice in a sterile isotonic buffered saline solution supplemented with an antifungal and an antibiotic drug.
   The process utilizes the following examplar components with a commercial source.
1) Hanks' Balanced Salt Solution —HBSS (SIGMA H8264)
2) Penicillin/Streptomycin (INVITROGEN 15140)
3) Amphotericin B-Amp B (SIGMA A2942)
4) Gentamicin (BIOWHITAKER BW17-518L)
5) Povidone-Iodine (CVS)
6) Dulbecco's Phosphate Buffered Saline—dPBS (SIGMA D8662)
7) Tissue Culture Water (SIGMA W3500)
8) 10×M199 Culture Media (INVITROGEN 11825)
9) GIBCO fetal bovine serum—FBS (INVITROGEN)
10) Sodium bicarbonate—NaHCO₃ (SIGMA S5761)
11) L-glutamine (INVITROGEN 25030)
12) Sodium fluorescein (NaFL) Powder (SIGMA F6377)
13) Agar (SIGMA A9045)
14) Porcine skin gelatin (SIGMA G1890)
15) 200 proof (100%) ethanol (SIGMA-Aldrich E7023)

Many changes can be made in the above-described invention without departing from the intent and scope thereof. It is therefore intended that the above description be read in the illustrative sense and not in the limiting sense. Substitutions and changes can be made while still being within the scope and intent of the invention and of the appended claims.

What is claimed:

1. A method for mammalian corneal excision from whole globe eyes, comprising:
procuring enucleated whole globe mammalian eyes from an abattoir and transporting said eyes on ice in an isotonic buffered saline solution supplemented with an anti-fungal drug;
incubating whole globe eyes in a broad spectrum antiseptic for approximately 2 minutes in a sterile field;
briefly rinsing said eyes with an isotonic buffered saline solution, and then incubating said eyes with an amino glycoside antibiotic in dPBS for 15 minutes;
excising the cornea from the eye with a scalpel using sterile technique, by making an incision 2-3 mm from the cornea into the sclera and cutting at this same distance all around the cornea until the cornea is free from the eye and removing the iris from the cornea with a pair of forceps and discarding said iris;
rinsing the cornea in a series of 12 sterile HBSS baths and storing said corneas in HBSS at room temperature until mounted; and
discarding any unacceptable corneas.

2. A method of claim 1, wherein said procuring isotonic buffered saline solution is Hank's Balanced Salt Solution (HBSS) and said anti-fungal drug is 5 μg/ml Amphotericin B, wherein said incubating broad spectrum antiseptic is 1% Povidone-iodine, and wherein said briefly rinsing isotonic buffered saline solution is dPBS and the following incubating amino glycoside antibiotic is 1 mg/ml Gentamicin.

3. A method for long-term mammalian corneal storage and preservation for greater than 21 days, comprising:
preparing, in a sterile manner, a 1.33% agar/gelatin mixture, autoclaving said mixture, and then storing said mixture at approximately 4° C. until needed;
warming a quantity of said 1.33% agar/gelatin mixture to approximately 60° C., and then cooling it to approximately 40-50° C., and then adding components to said agar/gelatin mixture to arrive at a plug mixture with the following components and the following final concentrations: agar/gelatin diluted to 1%; 10× M199 culture media diluted to 1×; 2.2 mg/ml NaHCO₃; 0.68 mM L-glutamine; 50 μg/ml Gentamicin; 1 μg/ml Amphotericin B; 100 units/0.1 mg/ml penicillin/Streptomycin; and tissue culture-grade water;
maintaining said plug mixture at approximately 40° C. until needed;
obtaining a quantity of recently excised mammalian cornea;
inverting each cornea in a respective well of a well plate filled with HBSS, so that the epithelium is bathed in HBSS below, and the endothelial cavity is exposed;
adding the added to, molten plug mixture drop by drop into the exposed endothelial corneal cavity, and then allowing said mixture to cool and congeal to form a plug;
placing the plugged corneas plug-side down in a tissue culture dish and filling with a custom corneal culture media to cover the sclera tissue surrounding the corneal tissue, but not the corneal tissue;
placing the tissue culture dish with said corneas into an incubator set to 37° C. and 5% CO₂ and periodically causing the custom culture media to flow over the corneas in culture to moisten and provide nutrients to the air-exposed corneal epithelial layer of cells; and
changing the corneal culture media daily using a sterile technique.

4. The method of claim 3, wherein said plug mixture is prepared in ultra pure sterile water and said mixture is storable for up to 6 months, wherein said quantity of agar/gelatin mixture selected for warming is less than 6 months old, wherein said well plate is a 24 well plate, wherein said tissue culture dish is 150×25 mm; wherein said custom corneal culture media is a customized M199 cornea culture media; wherein said periodic culture media flow is implemented by placing said tissue culture dish in a 45° rocker which periodically tilts the dish to 45°; wherein said changing of the corneal culture media daily uses a sterile technique for removing used, old corneal culture media from each culture dish by sterile aspiration and replacing it with pre-warmed, fresh corneal culture media, at approximately 37° C.; and wherein said daily changing is conducted for the duration of the corneal culture period.

5. A custom preservation tissue culture media, comprising:
approximately 90% by volume of M199 culture media;
approximately 10% by volume of fetal bovine serum;
approximately 2.2 g/L sodium bicarbonate;
approximately 0.68 mM L-glutamine;
approximately 100 units/0.1 mg/ml Penicillin/Streptomycin;
approximately 1-3 mg ml Amphotericin B; and
approximately 100 μg/ml Gentamicin.

6. A method of preparing a custom preservation tissue culture media by thoroughly mixing approximately 90% by volume of M199 culture media with approximately 10% by volume of fetal bovine serum, and adding and thoroughly mixing approximately each of 2.2 g/L sodium bicarbonate, 0.68 mM L-glutamine, 100 units/0.1 mg/ml penicillin/Streptomycin, 1-3 mg ml Amphotericin B; and 100 μg/ml Gentamicin, thereafter determining and adjusting the pH of the mixture to a range of 7.0-7.4, and then sterilizing the mixture by passing it through a 0.20 μm filter flask

7. A method for toxicological ocular irritation and recovery testing in excised cultured mammalian corneas, comprising:
obtaining a quantity of excised corneas;
placing the excised corneas in a dish of custom culture media;
allowing the excised corneas to equilibrate to the custom culture media conditions for approximately 24 hours;
periodically examining the culture media microscopically throughout the incubation period for the presence of contamination, and discarding contaminated corneas or treating those corneas with additional antibiotics/antifungal drugs when contamination is evident;
removing the cultured corneas from the incubator at the end of the incubation period and transferring them to a sterile field;
removing the culture media from the dish;
applying 10 μl or 20 mg of the toxicological irritant testing material directly to the corneal surface, and allowing the corneal tissue to be dosed with that testing material for 5 minutes;
rinsing individual corneas gently to remove the testing material;
transferring the rinsed corneas to a new, sterile, tissue culture dish and filling the new culture dish with custin corneal culture media covering the sclera tissue surrounding the corneal tissue, but not the corneal tissue itself;
placing the cornea and culture media containing dishes of in an incubator set to 37° C. and 5% CO₂ and periodically causing the custom culture media to flow over the corneas in culture to moisten and provide nutrients to the air-exposed corneal epithelial layer of cells;
periodically examining the custom culture media microscopically throughout the procedure for presence of contamination and discarding contaminated corneas or treating those contaminated corneas with additional antibiotics/antifungal drugs if contamination is evident; and
determining the toxicity of the test material by measuring cell vitality, death, or impairment in the cultured corneal tissue by one of the following techniques:
a) confocal microscopy paired with various vital dyes, whether colored or fluorescent;
b) reflective confocal microscopy using no dyes;
c) digital imaging techniques having image processing software; and
d) visual inspection combined with a vitality or death marker dye such as sodium fluorescein (NaFL) stain.

8. The method for toxicological ocular irritation and recovery testing of claim 7, wherein said individual cornea rinsing is conducted with approximately 2 ml of dPBS, wherein said new sterile tissue culture dish is 150×25 mm, wherein said custom corneal culture media is a customized M199 corneal culture media, wherein said periodic culture media flow over the cornea is implemented by placing said tissue culture dish on a custom rocker that periodically tilts to 45°, and wherein the measuring of cell vitality, death or impairment includes:
a) examining the culture media microscopically throughout the toxicological ocular irritation and recovery testing procedure for presence of contamination and discarding corneas or treating corneas with additional antibiotics/antifungal drugs if contamination is evident;
b) on days “1”, “2”, “3”, “7”, “10”, “14”, and “21” of procedure, determining the percentage range of damaged corneal area by staining damaged tissue with 2% sodium fluorescein (NaFL) stain using the following procedure:
i) removing cultured cornea dishes from the incubator, transferring to a sterile field, and adding sterile 2% NaFL drop-wise to each cornea until entire cornea is covered;
ii) repeating the NaFL application until all corneas in a dish are covered with the 2% NaFL solution and subsequently rinsing corneas gently with dPBS until excess NaFL is no longer present;
iii) individually transferring corneas with a disposable cell lifter to a new, sterile culture dish;
iv) observing NaFL stain retained in tissue that is damaged as a brown color by placing the culture dish containing the NaFL stained corneas on a regular white light box or by placing the corneas on a UV light box to observe NaFL stain retention;
v) scoring corneal damage by the indication of NaFL stain retention in damaged tissue by visually assessing the total area of damage per total corneal area and assigning a corneal damage score according to the following scale:
0=no corneal damage
1=0 to 25% corneal area damaged
2=25% to 50% corneal area damaged
3=50% to 75% corneal area damaged
4=75% to 100% corneal area damaged
or, alternatively, scoring corneal damage using a digital imaging system and image analysis software, such as METAMORPH [Nikon];
vi) filling each culture dish with M199 corneal culture media to cover the sclera tissue surrounding the corneal tissue, but not the corneal tissue itself;
vii) placing each dish of corneas in an incubator set to 37° C. and 5% CO₂ on a custom rocker that periodically tilts to 45° causing the culture media to flow over corneas in culture to moisten and provide nutrients to the air-exposed corneal epithelial layer of cells; and
viii) measuring repeatedly the damage caused by the toxin irritant testing material and control materials by determining cell vitality or death or impairment in the cultured corneal tissue at any required time during a culture period in excess of 21 days.

9. The method for toxicological ocular irritation and recovery testing of claim 8, also including the parallel processing of two tissue culture control dishes, a first with 100% concentration ethanol (positive control), and a second with dPBS (negative control).

10. A corneal culture method for human donor cornea preservation, comprising:
procuring enucleated whole globe human eyes from an eye bank and transporting said eyes on ice in an isotonic buffered saline solution such as Hank's Balanced Salt Solution (HBSS) supplemented with an anti-fungal drug;
in a sterile field, incubating the whole globe eyes in a broad spectrum antiseptic, briefly rinsing said eyes with an isotonic buffered saline solution, and then incubating said eyes with an amino glycoside antibiotic;
using a sterile technique, excising the cornea from each eye with a scalpel by making an incision 2-3 mm from the cornea into the sclera and cutting at this same distance all around the cornea until the cornea is free from the eye, and removing the iris from the cornea with a pair of forceps and discarding the iris;
rinsing the cornea in a series of 12 sterile HBSS baths and storing said corneas in HBSS at room temperature until mounted;
discarding any unacceptable corneas;
preparing, in a sterile manner a 1.33% agar/gelatin mixture in ultra pure sterile water, autoclaving said mixture, and storing said mixture at approximately 4° C. until needed;
warming a quantity of said agar/gelatin mixture, when needed, to approximately 60° C. to melt it, and cooling it to approximately 40-50° C., and then adding to the mixture to arrive at a plug mixture with the following components and the following approximate concentrations: agar/gelatin diluted to 1%; 10× M199 diluted to 1×; 2.2 mg/ml NaHCO₃; 0.68 mM L-glutamine; 50 μg/ml Gentamicin; 1 μg/ml Amphotericin B; 100 units/0.1 mg/ml penicillin/Streptomycin; and tissue culture-grade water;
maintaining said plug mixture at approximately 40° C. until all corneas are excised and ready to be plugged with the plug mixture;
inverting each cornea atop a respective well from a plate filled with HBSS so that the epithelium is bathed in HBSS below, and the endothelial cavity is exposed and able to be filled with the warmed molten added-to agar/gelatin mixture;
adding the warmed molten plug mixture drop by drop to the exposed endothelial corneal cavity, and then allowing said mixture to cool and congeal into a plug;
placing plugged corneas plug-side down in a tissue culture dish and filling with a customized M199 corneal culture media to cover the sclera tissue surrounding the corneal tissue, but not the corneal tissue;
placing culture dish with said corneas into incubator set to 37° C. and 5% CO₂ and periodically causing the customized culture media to flow over the corneas to moisten and provide nutrients to the air-exposed corneal epithelial layer of cells;
changing the corneal culture media daily using sterile technique; and
maintaining said corneas in said customized culture media incubation for up to four weeks;
wherein said customized M119 corneal culture media includes: approximately 90% by volume of M199 culture media; approximately 10% by volume of fetal bovine serum; and approximately of each of 2.2 g/L sodium bicarbonate: 0.68 mM L-glutamine; 100 units/0.1 mg/ml penicillin/Streptomycin; 1-3 mg/ml Amphotericin B; and 100 μg/ml Gentamicin.

11. The corneal culture method for human donor cornea preservation of claim 10, wherein said anti-fungal drug supplementing HBSS is approximately 5 μg/ml Amphotericin B, wherein said broad spectrum antiseptic in which said whole blobe eyes are incubated is 1% Povidone-iodine and wherein said incubation is for approximately 2 minutes, wherein said isotonic buffered saline solution in which said eye are briefly rinsed is dPBS; wherein said amino glycoside antibiotic in which said eyes are again incubated is 1 mg/ml Gentamicin in dPBS and wherein said again incubation period is for 15 minutes; wherein said well plate is a 24-well plate; wherein the warmed plug mixture is at approximately 40° C. when dropped into an endothelial corneal cavity; wherein said tissue culture dish is 150×25 mm; wherein said periodic culture media flow over the air-exposed corneal epithelial layer of each cornea is implemented by placing said tissue culture dish on a custom rocker that periodically tilts to 45°, and wherein the causing the culture media to flow, and wherein the daily changing of the corneal culture media includes removing used, old corneal culture media from each culture dish, by sterile aspiration and replacing with pre-warmed (37° C.) fresh corneal culture media.
